(19) United States
(12) Patent Application Publication
(10) Pub. No.: US 2002/0161416 A1
(43) Pub. Date: Oct. 31, 2002

(54) Nerves and muscle stimulator

(76) Inventor: Justine Huang, Palo Alto, CA (US)

Correspondence Address: OPPENHEIMER WOLFF & DONNELLY, P. O. BOX 10356, PALO ALTO, CA, 94303, US

(21) Appl. No.: 10/133,892

(22) Filed: Apr. 26, 2002

Related U.S. Application Data
(60) Provisional application No. 60/287,085, filed on Apr. 26, 2001.

(51) Int. Cl. A61N 1/18

(52) U.S. Cl. 607/48

(57) ABSTRACT

A muscle stimulator for stimulating a person's facial or neck muscle contraction and increase circulation for collagen and elastic fiber regeneration. The stimulator has a control unit that includes at least one electrical receptacle, a display, at least one control button, and a power source for providing electrical power for muscle stimulation; and a mask having an index for fitting over a person's face, including a plurality of electrodes for providing stimulation to the person's muscle, wherein the index providing a reference to align electrodes with stimulation points on the person's face.

### Cross-Reference to Related Applications

Reference is made to and priority claimed from U.S. Provisional Application Ser. No. 60/287,085, filed Apr. 26, 2001, entitled “Nerves and Muscle Stimulator.”

### BACKGROUND OF THE INVENTION

1. Field of Invention

This invention relates to devices for stimulating muscle contraction and increase circulation for collagen and elastic fiber regeneration of a human body.

2. Description of the Prior Art

Traditionally muscle stimulators includes a plurality of standalone electrical trigger dots to be attached to a person's body. Technician who attends the stimulation operation needs to know where on the person's body to attach the trigger dots. Further, the traditional muscle stimulators, due to the plurality of trigger dots, have electrical wires tangling to each other and are not very tidy. Since such trigger dot can be used in any section of the person's body, they cannot have independent control for triggers to be used on different sections of the person's body.

It is desirable to have a muscle stimulator that does not have all the wires with trigger dots to be attached to a user.

### SUMMARY OF THE INVENTION

Briefly, a presently preferred embodiment of the present invention includes a muscle stimulator for stimulating a person's facial or neck muscle contraction and increase circulation for collagen and elastic fiber regeneration. The stimulator has a control unit that includes at least one electrical receptacle, a display, at least one control button, and a power source for providing electrical power for muscle stimulation; and a mask having an index for fitting over a person's face, including a plurality of electrodes for providing stimulation to the person's muscle, wherein the index providing a reference to align electrodes with stimulation points on the person's face.

In yet another embodiment of the present invention includes a muscle stimulation mask. The mask includes an index for fitting over a portion of a person; and a plurality of electrodes for providing stimulation to the person's muscle, wherein said index providing a reference to align electrodes with stimulation points on the person's face.

In an alternative embodiment of the present invention includes a method for simulating muscles. The method includes providing a control unit having at least one electrical receptacle, a display, at least one control button, and a power source for providing electrical power for muscle stimulation; and providing a mask having an index for fitting over a person's face, including a plurality of electrodes for providing stimulation to the person's muscle, wherein the index providing a reference to align electrodes in the mask with stimulation points on the person's face.

The foregoing and other objects, features, and advantages of the present invention will be apparent from the following detailed description of the preferred embodiment which makes reference to the several figures of the drawing.

### IN THE DRAWINGS

FIGS. 1a-1g are diagrams illustrating exemplary parts of the muscle stimulation device of the invention;

FIG. 2 is a human facial diagram with various exemplary masks fitting over the human's face and neck;

FIG. 3 is a human facial diagram identifying some exemplary trigger points on a human face and neck for muscle stimulation purposes;

FIGS. 4-6 are diagrams of various exemplary masks with trigger points embedded therein for muscle stimulation purposes; and

FIG. 7 is an alternative embodiment of the control unit of the present invention.

### DETAILED DESCRIPTION OF THE PREFERRED EMBODIMENTS

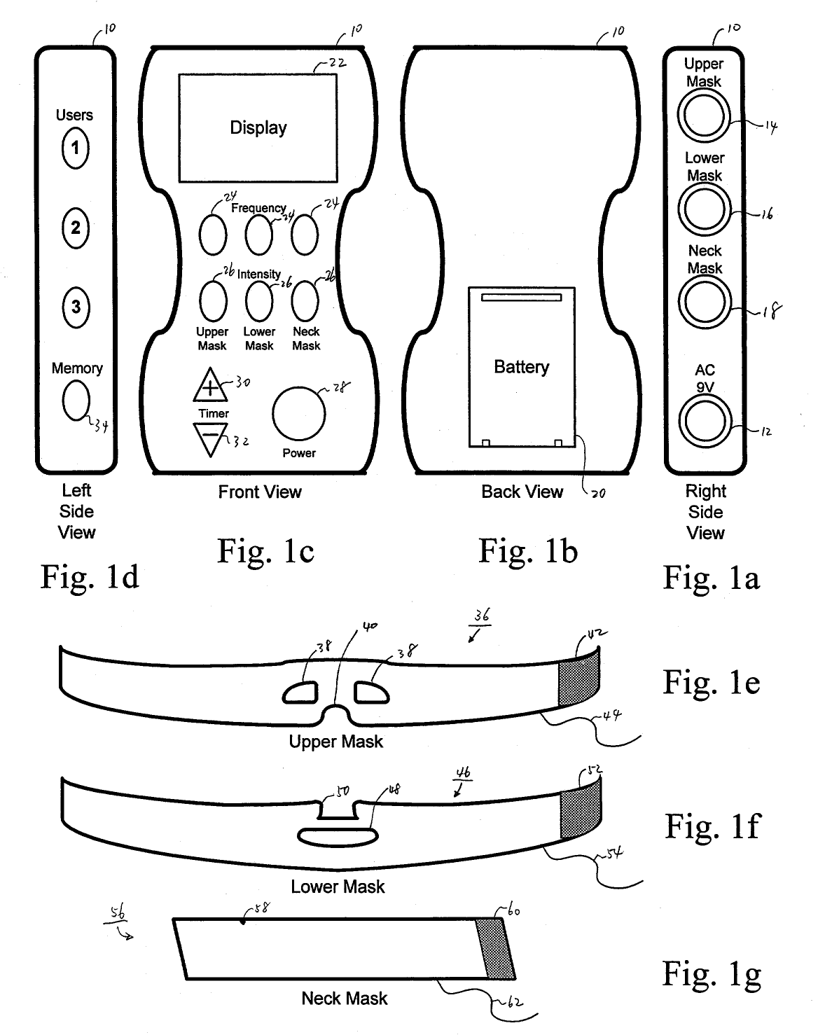

The present invention relates to devices for stimulating muscle contraction and increase circulation for collagen and elastic fiber regeneration of human body. FIGS. 1a-1g are diagrams illustrating exemplary parts of the muscle stimulation device of the present invention. The stimulation device includes a control unit 10 as illustrated in FIGS. 1a-1d and a plurality of masks 36, 46, 52, as illustrated in FIGS. 1e-1g. Each of the mask 36, 46, 52 is for stimulating different portions of a human face or neck. The control unit 10 provides electronic pulses to be distributed to the mask to stimulate the muscle or skin of the user. The device includes one control unit 10, a power supply, and three regional facial masks with a total of 42 electrodes corresponding to 42 to trigger points on a human's face and neck.

FIG. 1a is a diagram illustrating the right side view of the control unit 10. The control unit has a power receptacle 12 for receiving a plug from a power supply (not shown). In the preferred embodiment, it is desired that the power supply provides a 3-volt alternating current (AC) power to the control unit 10. The control unit 10 further includes a plurality of electrical receptacles 14, 16, and 18, wherein each one is equipped to electrically couple to a corresponding mask. In the exemplary embodiment, the receptacles were identified as upper mask 14, lower mask 16, and neck mask 18 respectively and their functions are described below. These receptacles can be operated individually independent to one another or simultaneously.

FIG. 1b is a diagram illustrating the back side of the control unit 10. The control unit 10 includes a receptacle 20 wherein a battery may be placed to provide the power to the stimulation device. The battery receptacle 20 is equipped with a cover to prevent the battery from falling out of the control unit. In a preferred embodiment of the present invention, the control unit 10 requires two AA-sized battery to operate.

FIG. 1c is a front view of the control unit 10. The control unit 10 includes a display 22, which can be a liquid crystal display (LCD) or a display composed of light-emitting diodes (LEDs). The display 22 is for displaying the strength of stimulation frequency and intensity, the location of skin under treatment, and remaining time for treatment as well as battery life. It further includes a plurality of frequency selection buttons 24 for selecting the frequency of the electronic pulse for the treatment and a plurality of pulse intensity selection buttons 26 for selecting the intensity of the electronic pulse for the treatment. Each of the plurality of frequency selection buttons 24 and intensity selection buttons 26 are designated for the different area on the human body of treatments, wherein an upper mask is for treating the upper facial of a human covering the forehead and eyes area of the face, a lower mask is for treating the lower facial of a human covering the cheek and chin of a human, and the neck mask is for treating the neck of a human. These buttons controls the output current and frequency of each of the receptacles so that different intensity, frequency, and duration can be applied to the mask coupled to the receptacle. In one embodiment of the present invention, each of the frequency and intensity to each mask has a predetermined default setting and level of increment for adjustment via pushing the respective button. Each of the frequency and intensity further has a predetermined range of operation, the frequency ranges from 10 to 50 Hz and the intensity has a current amplitude of 10 mA to 100 mA, and generates rectangular biphasic, exponentially decaying, triangular monophasic stimulation waveform. The adjustment to settings are arranged in a revolving manner so that continuing increasing the setting beyond the maximum allowable range will revolve to the minimum allowable range. The present readings of the intensity and frequency will be provided on the display through out the process of adjusting operation. In an alternative embodiment, each button controls two switches through its upper and lower edges, wherein the upper edge switch increases the setting and the lower edge switch decreases the setting. The control unit 10 further includes a power button 28 for activating or deactivating the device. The control unit 10 also has two timer control buttons 30, 32 one is marked with a “+” and the other marked with a “−” sign thereon for increasing or decreasing the duration of treatment. The timer can be set up to fifteen minutes for each treatment.

FIG. 1d is a left side view of the control unit 10. A memory button 34 is provided to memorize the preferred setting of time duration, frequency, and intensity of a particular setting. Three different setting can be memorized by the control unit 10 as identified by the users 1, 2 and 3 buttons. The user pushes the memory button follow by one of the three user button to memorize the setting under that particular number. To recall the setting simply push the user number button.

FIG. 1e is a diagram illustrating an upper mask 36. It has two holes 38 in the middle of the mask for fitting over the user's eyes, a notch 40 that fits right over the user's nose, a fastening 30 means 42 for tightening the mask around the user's upper face and forehead. In an exemplary embodiment, the fastening means may be a velcro, buttons or pins. The upper mask 36 further includes an electricity wire 44 that has a connector (not shown) for connecting to one of the receptacles 14, 16, 18 of the control unit 10. In the exemplary embodiment, the connector is to be connected to receptacle 14 identified as the upper mask. The notch 40 provides a reference so that electrodes in the upper mask 36 align with the trigger points on the user's face.

FIG. 1f is a diagram illustrating a lower mask 46. It has one hole 48 in the middle of the mask for fitting over the user' mouth, a notch 50 that receives the user's nose so that the nostrils of the user are not covered. It further includes a fastening meanings 52 for tightening the mask around the lower facial of the user and the cheek and chin. In an exemplary embodiment, the fastening means may be a velcro, button or pins. The upper mask 26 further includes an electricity wire 54 that has a connector (not shown) for connecting to one of the receptacles 14, 16, 18 of the control unit 10. In the exemplary embodiment, the connector is to be connected to receptacle 16 identified as the lower mask. The notch 50 provides a reference so that electrodes in the lower mask 46 align with the trigger points on the user's face.

FIG. 1g is a diagram illustrating a neck mask 56. The neck mask 56 includes an index 58 that should be position below the user's right ear upon fitting it over the user's neck. It further includes a fastening meanings 60 for tightening the mask around the user's neck. In an exemplary embodiment, the fastening means may be a velcro, button or pins. The neck mask 56 further includes an electricity wire 62 that has a connector (not shown) for connecting to one of the receptacles 14, 16, 18 of the control unit 10. In the exemplary embodiment, the connector is to be connected to receptacle 18 identified as the lower mask. The index 58 provides a reference so that electrodes in the neck mask 46 align with the trigger points on the user's neck.

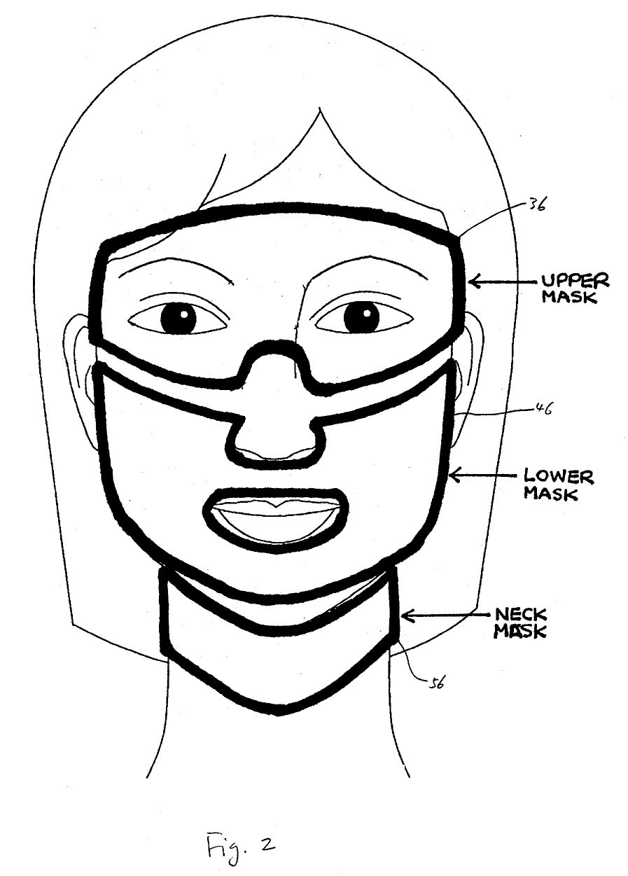

FIG. 2 is a human facial diagram with various exemplary masks fitting over a person's face and neck. The upper mask 36 is fitting over a person's upper face including the forehead. The lower mask 46 is fitting over the person's lower face including cheek and chin. The neck mask 56 is fitting over the person's neck. Each mask comes in different sizes to accommodate most human head size for best alignment of electrodes to user's facial trigger points. Moreover, each mask provides with an adjustable closure for precise fitness.

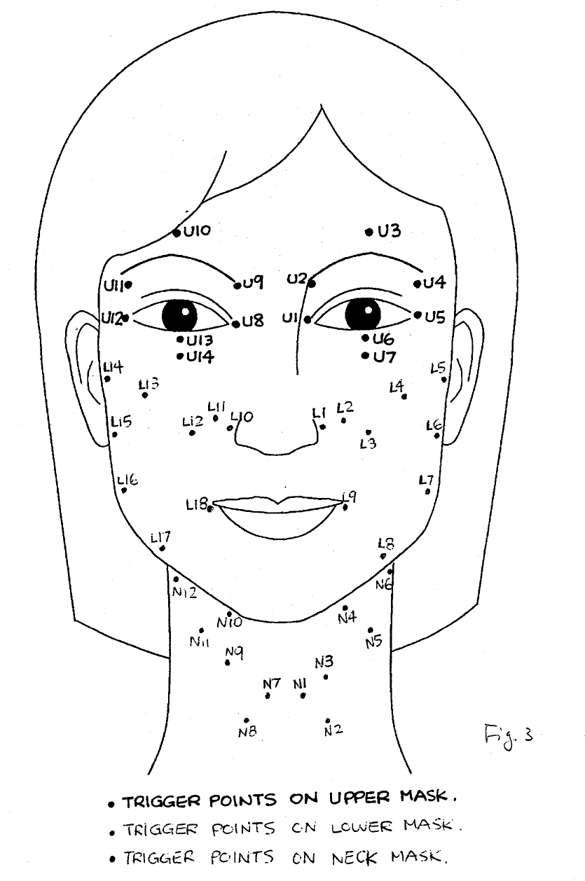

FIG. 3 is a human facial diagram identifying some exemplary trigger points on a human face and neck for muscle stimulation purposes. The trigger points are identified with a leading alphabet “U”, “L”, or “N” indicating as being covered by the upper mask, lower mask, or neck mask respectively. Fourteen trigger points are identified in the upper mask area, eighteen trigger points are identified in the lower mask area, and 12 points are identified in the neck mask area. These trigger points are acupuncture points on a human face or neck.

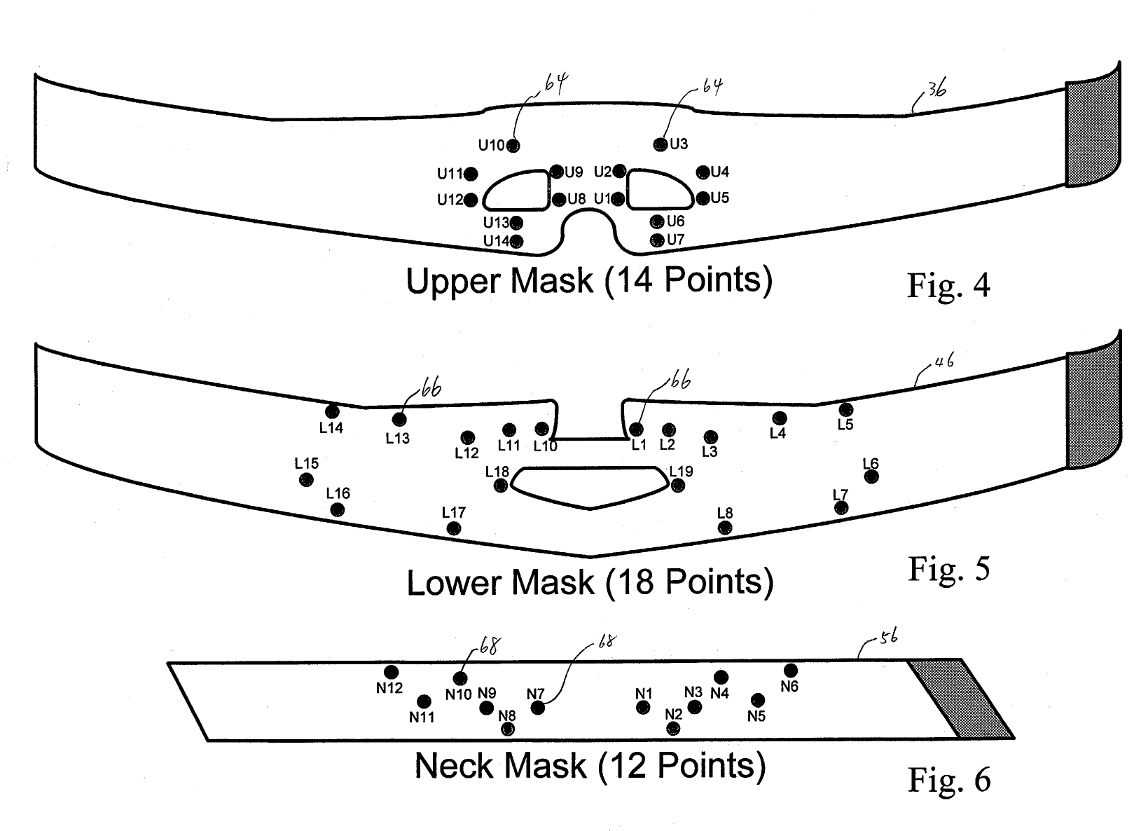

FIGS. 4-6 are diagrams of various exemplary masks with electrodes embedded therein for nerve and muscle stimulation purposes. The upper, lower, and neck masks are silicon over-all masks for its electricity conductivity. The upper mask 36 includes 14 electrodes 64 corresponding precisely to the trigger points on the upper face of the user. The electrodes are made of metal to stimulate underlying nerves and muscle contractions to produce more blood and oxygen for the renewal of both muscle and skin. In the preferred embodiment, metal electrodes 64 transmit mild electrical current under three volts to stimulate facial nerves and innervate underlying facial muscles. The electrodes 64, while having current running through it from the control unit 10, stimulate epidermis to generate new cell growth for a healthy and tight facial skin. The lower mask 46 includes 18 electrodes 66 corresponding precisely to the trigger points on the lower face of the user. The neck mask 56 includes 12 electrodes 68 corresponding precisely to the trigger points on the neck of the user. The functionality of the electrodes and the lower or the neck mask is similar to that of the upper mask and the description above for the upper mask applies to the lower mask as well.

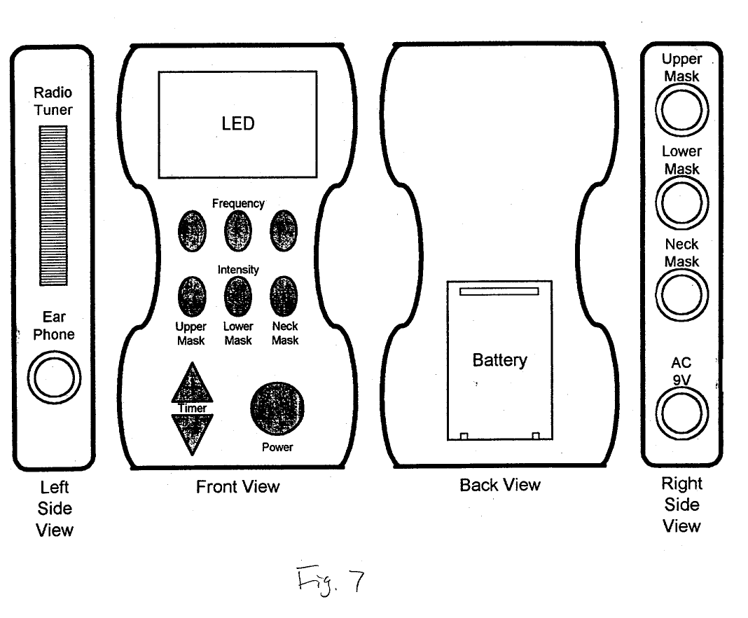

FIG. 7 is an alternative embodiment of the control unit of the present invention. In this embodiment, the memory feature has been replaced with a radio tuner 70 and a receptacle for headphone 72. User may listen to the radio station of their choice while using the nerves and muscle stimulator of the present invention.

In yet another alternative embodiment, the electronic pulse is substituted with an ultrasonic device. The intensity of the ultrasound is between 10 mW/cm³ and 100 W/cm³, and frequency is within 1 to 10 MHz range of applicable ultrasound to human body.

The electronic pulses or ultrasonic waves applied on acupuncture points on a human facial or neck muscle of the present invention is capable to stimulate muscle contraction and increase circulation for collagen and elastic fiber regeneration. The effect is it smoothes the person's forehead wrinkle by stimulating frontalis muscle through temporal nerve branches. It removes puffiness of undereye bags by stimulating orbicularis oculi muscle through zygomatic infratrochlear, and supratrochllear nerve branches, and partially through temporal nerve branch. It further removes crow's feet by stimulating orbicularis oculi muscle through zygomatic nerve branch and partially through temporal nerve branch. It removes hollowness under the eyes by stimulating orbicularis oculi muscle through zygomatic nerve branch and partially through temporal nerve branch. Moreover, it improves smile lines and sagging cheek muscle by stimulating buccinator muscle through infraorbital, buccal and zygomatic nerve branch VII. It reduces at the corners of a droopy mouth by stimulating orbicularis anguli oris muscle. It improves double chin and sagging chin line by stimulating depressor anguli oris muscle and masseter muscle through marginal mandibular nerve branch. It also smoothes neck and jawline by stimulating platysma muscle.

While the invention has been particularly shown and described with reference to the preferred embodiments, it will be understood by those skilled in the art that many other modifications and variations may be made thereto without departing from the broader spirit and scope of the invention as set forth in the claims. The specification and drawings are accordingly to be regarded as illustrative, rather than in a restrictive sense.

What is claimed is:

1. A muscle stimulator, comprising:
a control unit having at least one electrical receptacle, a display, at least one control button, and a power source for providing electrical power for muscle stimulation; and
a mask having an index for fitting over a person's face, including a plurality of electrodes for providing stimulation to the person's muscle, wherein the index providing a reference to align electrodes with stimulation points on the person's face.

2. A muscle stimulator as recited in claim 1, wherein said power source providing power to generate electronic pulses as a source for muscle stimulation.

3. A muscle stimulator as recited in claim 1, wherein said power source providing power to generate ultrasonic waves as a source for muscle stimulation.

4. A muscle stimulator as recited in claim 1, wherein said control button is for selecting frequency of the stimulation source.

5. A muscle stimulator as recited in claim 1, wherein said control button is for selecting intensity of the stimulation source.

6. A muscle stimulator as recited in claim 1, wherein said control button is for selecting duration for the stimulation.

7. A muscle stimulator as recited in claim 1, wherein said mask is a silicon over-all mask.

8. A muscle stimulator as recited in claim 1, wherein said electrodes are made of metal.

9. A muscle stimulator as recited in claim 1, wherein said mask is for fitting over a person's neck.

10. A method for simulating muscles, comprising:
providing a control unit having at least one electrical receptacle, a display, at least one control button, and a power source for providing electrical power for muscle stimulation; and
providing a mask having an index for fitting over a person's face, including a plurality of electrodes for providing stimulation to the person's muscle, wherein the index providing a reference to align electrodes in the mask with stimulation points on the person's face.

11. A method for simulating muscle as recited in claim 10, further comprising:
generating electronic pulses to stimulate muscles.

12. A method for simulating muscle as recited in claim 10, further comprising:
generating ultrasonic waves to stimulate muscles.

13. A method for simulating muscle as recited in claim 10, further comprising:
selecting frequency for said stimulation.

14. A method for simulating muscle as recited in claim 10, further comprising:
selecting intensity for said stimulation.

15. A method for simulating muscle as recited in claim 10, further comprising:
selecting duration for said stimulation.

16. A method for simulating muscle as recited in claim 10, further comprising:
providing a silicon over-all mask.

17. A method for simulating muscle as recited in claim 10, further comprising:
providing a metal electrodes in said mask.

18. A muscle stimulation mask, comprising:
an index for fitting over a portion of a person;
a plurality of electrodes for providing stimulation to the person's muscle, wherein said index providing a reference to align electrodes with stimulation points on the person's face.

19. A muscle stimulator mask as recited in claim 18 is a silicon over-all mask.

20. A muscle stimulator mask as recited in claim 18, wherein said electrodes are made of metal.

21. A muscle stimulator mask as recited in claim 18, wherein said mask is for fitting over a person's neck.

22. A muscle stimulator mask as recited in claim 18, wherein said mask is for fitting over a person's face.
